(19) United States
(12) Patent Application Publication
(10) Pub. No.: US 2009/0017095 A1
(43) Pub. Date: Jan. 15, 2009

(54) COMPOSITION FOR FILLING A BONE DEFECT

(75) Inventors: Laurence Barnouin, Venerieu (FR); Laurent Laganier, Vernaison (FR)

Correspondence Address: CANTOR COLBURN, LLP, 20 Church Street, 22nd Floor, Hartford, CT, 06103, US

(73) Assignee: TBF - BANQUE DE TISSUS, BRON (FR)

(21) Appl. No.: 11/909,030

(22) PCT Filed: Oct. 24, 2005
(86) PCT No.: PCT/FR2005/002651

§ 371 Date: May 8, 2008

(30) Foreign Application Priority Data
Oct. 22, 2004 (FR) 0411271

(51) Int. Cl. A61F 2/28; A61K 35/32

(52) U.S. Cl. 424/426; 424/549

(57) ABSTRACT

The invention relates to a composition for filling a bone defect, characterized in that it comprises allogenic bone powder, calcium sulfate, a binder selected from starch or a starch derivative and a mixing solution. The invention more particularly relates to a composition for filling a bone defect, characterized in that the starch derivative is hydroxyethyl starch.

The filling of bone cavities is a frequent problem in orthopedic surgery. Different surgical techniques and a range of products may potentially be used by surgeons depending on the deficiencies to be filled and the origin thereof, which may be due to reconstructive surgery and the putting in of plates, screws or prostheses, or may be pathological and due, for example, to tumors.

Bone filling products may be of human, animal, mineral or synthetic origin. All these materials must be biocompatible (that is to say: non-toxic, non-immunogenic and non-carcinogenic), must promote or induce bone regrowth, be resorbable over the medium term and be storable and available. The choice of material is influenced by the amount to be filled, the site, the local conditions and the goal being pursued.

This invention thus relates to the area of compounds intended for improvement of bone tissue repair and for filling bone deficiencies or defects.

Bone tissue is made up of a matrix of connective tissue to which are attached mineral elements that ensure its rigidity. Two types of structure may be distinguished: compact bone and spongy bone, formed from bone trabeculae between which there is located hematopoietic or fatty tissue. In both cases, the bone is formed from superimposed lamellae, several microns thick, the orientation of which follows that of the collagen fibers.

The distribution of these two types of bone is different depending on the skeletal location: spongy bone is in the majority at the level of the vertebral bodies, the radial epiphysis and the calcaneum, whereas cortical bone is found at the level of the posterior vertebral arcs, the shafts of the long bones and the neck of femur.

This specialized connective tissue is made up of an organo-mineral matrix and of cells. 70% of the bone tissue matrix is formed of the mineral phase, 20% of organic components and 10% of water. This breakdown by weight is only approximate, since the nature of component elements of the bone remains more or less constant, while their proportion may vary considerably, depending on the nature of the bone, sex, diet and age.

The mineral component of the bone includes the following elements:

Calcium (percentage by mass: 36.7%), Phosphorus (16.0%), Carbonate (8.0%), Sodium (0.77%), Magnesium (0.46%) and Fluorine (0.04%).

The crystalline phase of bone resembles hydroxyapatite (Ca₁₀(PO₄)₆(OH)₂), the mesh of which is hexagonal. Apatite is a host structure that is able to incorporate a significant number of chemical elements or allow lacunae to appear.

90% of the organic matrix is formed of Type 1 collagen, 10% of other non-collagenic proteins, primarily osteocalcin and osteonectin. This matrix is degraded and synthesized, respectively, by two types of cells: osteoclasts and osteoblasts. The activity of these two cells controls the dynamic process called “remodeling” which, over the course of life, replaces the tissues of the bone matrix and allows preservation of their biomechanical properties.

There are two principal types of bone cells: osteoblasts and osteoclasts.

Osteoblasts are at the origin of bone tissue genesis. They are located on the surface of bone tissue, most often bordering a tissue in the process of being formed: osteoid tissue.

Osteoblast differentiation corresponds to a complex process bringing into play multiple interactions among the cells and the matrix. After an initial stage of proliferation of precursor cells, an osteoblast progressively acquires the characteristics of a functionally differentiated cell. Its essential function is to synthesize and mineralize the organic bone matrix, essentially composed of collagen, non-collagenic proteins and growth factors.

Moreover, cells of the osteoblast series play an important role in controlling bone remodeling, on the one hand through their capacity for synthesizing numerous growth factors, on the other inasmuch as they are target cells for hormones that control osteoclast differentiation.

Bone formation essentially depends on the number of osteoblasts more than on the activities among each of them. Moreover, the importance of the cellular proliferation stage has been shown, beginning with the osteoblast precursors, in controlling normal and pathological bone formation, and the essential role of the growth factors affecting recruitment and proliferation of osteoblasts has been underscored.

Once the bone tissue has been formed, the osteoblasts find themselves trapped there. These cells are then called Osteocytes.

Osteoclasts are responsible for the process of bone resorption. These are multinucleate cells quite large in volume. Osteoclasts are located at the surface of the bone at the level of the resorption areas. Bone resorption is made possible by a significant enzyme content. The mineral portion is dissolved by a local lowering of the pH to the vicinity of 5, due to the extracellular release of protons by the action of carbonic anhydrase. The degradation of the organic matrix takes place through enzymatic fission by the action of cathepsins and hydrolases.

The two types of bone cells, osteoclasts and osteoblasts, derive from the hematopoietic stem cells and the stromal bone marrow cells, respectively.

Three mechanisms contribute to the depositing of bone after a bone graft: osteogenesis, osteoconduction and osteoinduction.
osteogenesis translates into the formation of bone from the implanted osteoblasts.
osteoconduction consists of 3 stages: infiltration of the graft by the host osteoblasts, their proliferation, then new bone formation in a favorable environment (autologous bone scaffolding and Hydroxyapatite matrix).
osteoinduction corresponds to new bone formation from mesenchymatous cells of the recipient, which differentiate into osteoblasts under the influence of the growth factors present in the matrix.

Four essential elements are necessary to permit bone regeneration:
the osteoconductive matrix,
the osteoinductive factors, that is to say, the chemical agents which induce the different stages of bone repair,
the osteogenic cells, which may be differentiated and facilitate cell repair,
structural integrity.

In the case of a spongy autograft, complete revascularization takes place in from two to three weeks. The osteoblasts of the graft at the recipient site directly deposit osteoid tissue on the trabeculae, with graft-bone consolidation being accomplished in from five to six weeks. In the case of a cortical graft, a preliminary phase of osteoclastic resorption is necessary, and consolidation is achieved only starting with the third month.

Hence, an autograft possesses the four elements needed for bone regeneration.

In the case of an allograft, the allogenic bone does not possess any osteogenic property in itself, that is to say, it is incapable on its own of giving rise to the formation of new bone. Its essential property is to guide the bone re-growth coming from the bone bed in which it is placed. The extent of the graft-host contact surface is determinative of the speed of graft penetration. Grafts totally fitted into a bone cavity have a more favorable environment for osteogenesis than those that are simply placed in apposition.

An allograft thus possesses only two of the elements needed for bone regeneration.

Bone pathologies requiring filling compounds according to the invention are pathologies such as osseous tumors.

Benign tumors, which are much more frequent that malignant tumors, are of different histological types (osteoid osteoma, exostosis, fibroma . . . ) and may eventuate in grafts for filling purposes.

Numerous orthopedic (or dental) surgeries require plates, screws or prostheses to be put in. In order to facilitate the integration of these devices into the bone site, the interstices may be filled with the help of a bone graft or a bone substitute.

At the present time, it is this type of surgery which most often requires bone filling compounds.

The ideal bone-filling product is a biomaterial having properties close to bone, with the ability to be simultaneously safe, effective and available:

The safety of a biomaterial rests upon its biocompatibility, that is to say, on the lack of toxicity of this biomaterial and the products of its degradation. Evaluation of this safety is essential, and needs to be carried out before animal studies of its biofunctionality. It is based on the application of acknowledged standards. In addition to studies of its mutagenic and carcinogenic character, research on the host's reactions to the biomaterial requires an in-depth analysis of the inflammatory reaction (presence of giant cells, interposition of connective tissue between biomaterial and host tissue . . . ).

The effectiveness of a bone substitute depends on its short, medium and long-term clinical results.

The study of the biomaterial osteoconductive and, possibly, osteoinductive qualities is of interest in predicting its clinical effectiveness.

Osteoconduction corresponds to the “passive property of a material for receiving the bone regrowth, through vascular and cellular invasion starting from the recipient bone tissue in contact with the material.” Osteoconduction is in part dependent on the size of the biomaterial pores.

Osteoinduction is the capacity to promote bone re-growth by inducing the bone metabolism of the recipient bone site.

Availability of a bone substitute supposes that it can be made in sufficient quantity to respond to all demands, that it can be preserved for a sufficiently long time without significant degradation of its essential properties and that the conditions for its preservation are extremely simple and able be met by any department of osteosurgery.

The bone fillers utilized today are diverse and varied in nature and composition. Two principal types of bone fillers can be distinguished: bone grafts and bone substitutes.

The term bone graft designates a contribution of free bone tissue, living or dead. There are two categories: autografts and allografts, excluding bone filling compound products containing bone and another product.

An autograft is the oldest filling material still currently in use by orthopedic surgeons. Autografts are the reference material for bone substitutes. An autograft, by definition, is taken from the patient at the level of a donor site (hip, cranium . . . ) and is placed at the same operating time at the place where the filling is necessary. It is the most osteogenic of the filling materials: osteoinductive and osteoconductive.

An allograft designates a graft taken from a human being who is not the recipient.

As of this date, different approaches are employed to preserve allografts. The graft may be either simply frozen, freeze-dried or washed. Additional sterilization is advised, through physical (ionizing irradiation) or chemical (ethylene oxide) means, or using heat. The aim of these treatments is to reduce the antigenicity of the bone tissue without thereby excessively altering its biological and mechanical properties.

When a graft is not possible or desirable, surgeons have recourse to bone substitutes.

Among known bone substitutes one may note xenografts, which designate a graft of animal origin. The most common is prepared from the bone of an adult bull.

The difficulties tied to preparing such products reside in obtaining the indispensable biological safety and preserving the integrity of the bone's biphasic structure (collagenous and mineral).

Derivatives of coral are also used, possessing a totally porous structure which favors bone penetration. They have been used for a long time.

Phosphocalcic or ionic cements form a new class of bone substitutes characterized by setting and hardening in a wet medium. However, their objective is essentially the sealing of prostheses and not the filling of bones.

Calcium phosphate ceramics are likewise used since the crystals of biological apatites, principal constituents of bones and teeth, belong to the family of calcium phosphates. They form the mineral portion of the bone and could act as precursors at the time of mineralization.

Mineral substitutes, with a calcium sulfate or even a synthetic resin base, are also available.

There are other bone substitutes. These are made up of two phases (allogenic bone and another product), such as, for example, mixtures of calcium sulfate and demineralized bone, glycerol and demineralized bone, gelatin and demineralized bone, sodium hyaluronate and demineralized bone, or a copolymer and demineralized bone, in the form of a paste prepared on the spot or a ready to use paste.

Problems remain because a bone filling compound, an ideal filling material allowing for all situations to be addressed, does not exist. A filling will not be envisaged in the same way after excision of a tumor, after traumatic impairment of a limb, whether in the acute phase or in the stage of pseudo-arthrosis, after a prosthesis is unsealed, after an additional osteotomy, or in the treatment of extended scolioses.

Filling materials are of significant practical interest since filling bone defects is necessary in multiple situations, such as, for example, a tumorectomy, the post-tumorectomy filling, the filling of non-tumorous and aseptic spongy defects, reconstruction of cortical bone and vertebral body surgery.

These highly varied indications require extended mechanical properties and a different consistency and profile.

Numerous publications and patents describe compounds like those mentioned above. For example, from U.S. Pat. No. 6,030,635 we learn of a compound with a base of hydrogels chosen from among sodium hyaluronate and chitosans, allowing for compounds to be prepared containing only from 1 to 3% gel, and from WO-A-9639203 we learn of collagen compounds containing particles of demineralized bone.

From US2002/0110541, we learn of a compound containing calcium sulfate, a mixing solution and a plasticizing substance made up of a cellulose derivative such as carboxymethyl cellulose, forming a mucoadhesive, biodegradable hydrogel that is able to form a filling product which shows significant mechanical resistance and is able to be used alone. See the paragraph “robustness” in the description.

From Ruskin, J. D. et al., Clinical Oral Implants Research, April 200 [sic; year incomplete], vol. 11, 2, pp. 107-115, one learns of the preparation of a compound containing calcium carbonate, hydroxyethyl starch and a growth factor, for testing osteogenesis and bone regeneration in buccal bone lesions.

From Pietrrzak, W. S. et al., Journal of Craniofacial Surgery, July 2000, vol. 11, 4, pp. 327-333, one learns of the interest in using calcium sulfate as an in vivo filling product.

However, none of the products currently described and/or marketed allows for all of the criteria set out above to be satisfied.

The present invention has permitted compounds to be made allowing for all the situations described to be addressed, while retaining properties satisfying all of the criteria.

In fact, in order to facilitate their use, these bone filling compounds, at the time they are used, i.e., during surgery, ideally need to come in the form of a malleable paste that does not need reheating, moldable if necessary to allow it to be put to use after being shaped, non-adhesive to instruments and gloves, but sufficiently cohesive and able to adhere to the tissues of the sites to which the paste will be applied. They must likewise be osteogenic.

The present invention hence concerns a bone filling compound characterized by the fact that it includes allogenic bone powder, calcium sulfate, a binder chosen from starch or a starch derivative and a mixing solution.

According to the invention, the starch derivative is chosen from among hydroxyethyl starch, carboxymethyl starch, starch glycolic acid, starch glycerol and pre-gelatinized starch.

It likewise concerns the bone filling compound according to the invention characterized by the fact that the starch derivative is hydroxyethyl starch.

According to the invention, the calcium sulfate is chosen from among di-hydrated, anhydrous or hemi-hydrated calcium, the defined crystalline form of which can be alpha, beta or both. It is preferably hemi-hydrated calcium sulfate.

The allogenic bone powder according to the invention is chosen from among bone powders, whether human or not, whether de-mineralized or not, which have undergone a viroinactivation treatment, with a particle size between 200 and 1600 μm.

It is preferably a human bone power which has undergone a viroinactivation treatment, with a particle size between 200 and 1600 μm.

According to the invention, the mixing solution is a physiologically compatible aqueous solution chosen from among an isotonic solution, physiological saline solution or a physiological liquid like serum or blood.

According to the applications, the aqueous solution may be buffered.

The latter shall be added to the mixture depending on the desired consistency of the final compound to with, a compound in the form of a paste that is malleable, injectable or able to be molded.

According to the invention, the allogenic bone powder will be incorporated in proportions between 0.1 and 80% v/v, the calcium sulfate in proportions from 50 to 300% m/o, the hydroxyethyl starch from 0.1 to 20% v/v and the mixing solution from 0.3 to 80% v/v.

The calcium sulfate exists in three forms: di-hydrated (CaSO₄-2H₂O), hemi-hydrated ((CaSO₄-½H₂O) and anhydrous (CaSO₄).

Di-hydrated calcium sulfate is the principal component of Gypsum.

It is from this mineral that plaster of Paris is made. After extraction, Gypsum is crushed, ground and dried. Subjected then to “cooking” (between 100 and 200° C.), it is partially dehydrated and yields plaster of Paris: hemi-hydrated calcium sulfate.

According to the mode of cooking employed, principally two forms of plaster are being researched:
the Alpha hemi-hydrated plaster, obtained by pressure-cooking in an autoclave.
the Beta hemi-hydrated plaster, obtained in a fluidized-bed oven or a direct-flame oven.

The di-hydrated and hemi-hydrated calcium sulfates have a difference in solubility at a temperature of 20° C. Thus, the addition of water to a powder of hemi-hydrated calcium sulfate will generate the formation of di-hydrated calcium sulfate, which will precipitate to give a hard stone. The crystallization of the gypsum translates into the setting of the plaster; it is accompanied by a characteristic rise in temperature, which may reach 37° C.

The quantity of water used for the setting of the plaster acts on 2 levels:
the rehydration water, which transforms the plaster back into gypsum or water of crystallization.
the excess water which contributes to setting the porosity value and which is eliminated upon drying.

The implanting of plaster of Paris in bone or tissue does not produce inflammatory reactions, it being well accepted by the body. But plaster of Paris by itself does not stimulate, but also does not inhibit, bone formation and it is absorbed and displaced very quickly from the implant site and the bone is rebuilt.

The bone powder permits bone regrowth at the level of the site filled. It is made of a mixture of calcified bone powder and demineralized bone powder. It simultaneously contributes calcium through the calcified bone powder and the growth factors needed for bone neoformation through the demineralized bone powder.

The bone may be of allogenic origin and may have come from the transformation of femur heads or solid bone of human origin, treated so as to inactivate viruses, then reduced in the form of chips.

The calcified bone powder may thus be obtained by direct grinding of spongy or cortical bone chips.

The role of the calcified bone powder is to contribute the minerals necessary for the process of new bone formation at the level of the site filled, without it being necessary to contribute any added mineral salts to activate this bone neoformation.

The demineralized bone powder is obtained by bone grinding and demineralization or by demineralization of bone fragments which are then ground.

The demineralized bone powder contributes the growth factors promoting bone neoformation that stimulate the osteoprogenitor cells.

The combination of the two bone powders, i.e., calcified bone powder and demineralized bone powder, permits stimulation of bone regrowth by osteoconduction and osteoinduction.

The particle size of the bone powders has an important role in the viscosity and consistency of the paste and will be between 100 and 1600 μm, and preferably between 200 and 500 μm for the calcified bone powder.

For example, PHOENIX® grafts are marketed by THF. Tissue banks may be used.

The aim of the procedure for transforming femur heads into PHOENIX® grafts is to clean the bone trabeculae and to use chemical solvents to inactivate the micro-organisms not detected by the blood test. This inactivation has been validated by the Institut Pasteur—Texcell on particularly resistant viruses. A stage of the procedure also permits protection against unconventional transmissible agents (prions).

The transformation procedure has been studied so as not to alter the intrinsic mechanical properties of spongy bone.

Calcium sulfate shows numerous characteristics that are of benefit to a bone substitute. It is inorganic, and hence all risks of virus or illness transmission are avoided. It is biocompatible, resorbable, osteoconductive; it serves as a medium for blood vessels and cells and permits revascularization. It constitutes a source of calcium, an essential element for bone reconstruction.

The hemi-hydrated form of calcium sulfate, more particularly, possesses favorable physical characteristics: it can be molded, is easily manipulable, possesses the ability to harden and is able to mix with other materials.

Starch is a polyoside sugar with the empirical formula (C₆H₁₀O₅)ₙ, n falling between 100 and 20,000. The chemical composition of the starch varies depending on its vegetable origin, but all starches include two polysaccharides, amylose and amylopectin.

Industrially, it is prepared from potato, wheat, corn and rice. Most corn, potato and wheat starches contain 70 to 85% amylopectin and 13 to 30% amylase.

Hydroxyethyl starch is starch onto which hydroxyethyl groups (C₂H₄—OH) are grafted. These hydroxyethyl groups may be attached to the glucose by the carbons located in positions 2, 3 and 6.

Hydroxyethyl starch is a pharmaceutical product in an injectable solution. It serves as a filling solution and a plasma substitute and does not form a solid hydrogel.

There are three parameters that permit the various types of hydroxyethyl starch to be identified:
molecular weight: hydroxyethyl starch is a macromolecule the molecular weight of which can go from 200,000 to 450,000 daltons. The lower its molar substitution rate, the more quickly is hydroxyethyl starch degraded by serum amylases.
degree of substitution: this is the number of hydroxyethyl group[s] per 10 molecules of glucose. This degree of substitution influences the product degradation time. The higher the degree of substitution, the longer the degradation time.
the C2/C6 ratio: this is the ratio between the number of hydroxyethyl groups attached to the carbon in position 2 and in position 6. Hydrolysis by the amylase is delayed more by a substitution in the carbon in position 2 than in that in position 6. Hence, the more this ratio increases, the longer will be the macromolecule degradation time.

The invention also concerns the use, for filling bone defects, of a bone filling compound characterized by the fact that it includes allogenic bone powder, calcium sulfate, a binder chosen from starch or a starch derivative, and a mixing solution.

More particularly, it concerns the use described above, characterized by the fact that the binder is hydroxyethyl starch.

According to the invention, the compound will be used either in the form of a paste that is more or less liquid, that is to say, malleable or injectable, or in the form of a pre-molded paste.

In a different mode of implementation when filling bone deficiencies of significant size, for example after tumor surgery, the compound according to the invention may itself be employed as a vehicle for the implanting of bone fragments.

As a function of the possible particular uses for the compound according to the invention, the compound according to the invention may also include one or more active pharmaceutical ingredients chosen, for example, from among antiviral agents, antibiotics, immuno-suppressants or anti-tumoral agents.

It may likewise include growth factors that will be released in situ, such as “Bone Morphing Proteins” like BMP 2 or 7 for the bone, promoting repair of the target tissues.

The invention likewise concerns the compound according to the invention in the form of a kit, to with, a container containing a compound which includes allogenic bone powder, calcium sulfate, a binder chosen from starch or a starch derivative and a container that holds the mixing solution.

When the compounds need to be molded for a particular use by the surgeon, for example to put osteotomy wedges in place, then the kit according to the invention also includes molds for osteotomy wedges.

Active pharmaceutical ingredients or growth factors may be added, either to the container containing the bone powder, or in a solution in the container that holds the mixing solution.

The invention will be better understood by reading the following sample embodiments:

### EXAMPLE 1

### Manufacture of Compounds According to the Invention

To manufacture a final volume of bone filling compound of around 3 cc requires:
1 cc of Phoenix bone powder, obtained from demineralized or non-demineralized bone powder which has undergone treatment to ensure its microbiological safety (viro inactivation)
4 g of calcium sulfate
0.1 g of hydroxyethyl starch
1.1 ml of physiological saline solution (aqueous solution of 0.9% NaCl)

The hydroxyethyl starch rate may vary as a function of the quality utilized: thus, an increase in the average molecular weight and the substitution rate cause the viscosity of the product to be increased.

The particle size of the bone powder used may cause a variance in the quantity of bone for the same volume of powder, thereby modifying the appropriate proportions of the other components. (particle size normally used: from 200 to 1600 μm).

The physiological saline solution may be replaced by another physiological solution. In particular, a buffer may be added.

Different formulations have been made.

| | | | Fraction of |
|---|---|---|---|
| | Osteotomy wedge | Paste | final volume |
| | 10.8 ml | 16.5 ml | (v/v or p/v) |
| Physiological saline | 4 ml | 6 ml | 20 to 80% |
| solution | | | |
| Bone powder (particle | 3.6 ml | 5.5 ml | 1 to 80% |
| size 20-3000 μm) | | | |
| Calcium sulfate | 14.4 g | 22 g | 40 to 300% |
| Hydroxyethyl starch | 0.36 g | 0.55 g | 0.5 to 20% |

After a sustained mixing lasting 3 minutes, this formulation allows a paste to be obtained of a consistency close to modeling paste.

The paste can be used for around 7 minutes before beginning to harden.

Setting requires a period lasting on the order of 15 minutes.

The product continues to harden after this time, until reaching a resistance on the order of 480 Mpa after drying. (hardness test sheet).

A slight rise in temperature is noted during setting. The product may reach a temperature on the order of 40° C. after vigorous mixing. This is still quite acceptable.

### EXAMPLE 2

### Molding and Manufacture of Osteotomy Wedges

Knee deformities such as Genu varum (with heels together, the inside faces of the knees remain separated by a greater distance the more pronounced the Genu varum is) and Genu valgum (conversely to Genu varum, the knees are curved inwards) very frequently entail a localized femorotibial arthrosis (inside or outside).

There is a surgical solution to remedy this: opening wedge osteotomy. The tibial or femoral bone is sectioned so as to be able to introduce an osteotomy wedge there (plates are fixed in place to avoid the wedge being crushed), which allows the angle of incline between the femur and the tibia to be modified and causes the deformity to disappear. The positioning of this osteotomy wedge must allow for bone reconstruction, for long-term preservation of the angular correction.

The present invention allows the surgeon, during the surgery, to choose the size of the osteotomy wedge so as to obtain the desired angle of correction, thanks to the particular way in which the bone filling compound, which may be molded, is presented.

The product is presented in the form of a kit which may contain some or all of the following components:
mold: a mold displaying cavities corresponding to osteotomy wedges of different sizes (variation of the dimensions and/or form)
bone filling product: the compound according to the invention which has a hardening time of around 15 minutes, for example the compound in Example 1.

After mixing, the compound obtained is in the form of a paste malleable for around 10 minutes. It is during this period of time that the product may be molded into the form of an osteotomy wedge with the aid of a mold.

After around 15 minutes, the paste hardens. The osteotomy wedge may then be removed from the mold and used by the surgeon.

The time of hardening was evaluated through comparative tests. The latter made it possible to note that mixing time has an influence on the time of setting.

### EXAMPLE 3

### Hardness Tests

Formulations according to the invention prepared in accordance with Example 1 are placed in sealing cups¹ for at least one hour. These constitute the “test pieces.”¹ Translator's Note: The context in unconfirmed.

The material for testing hardness is as follows:
JFC-TC3 compression machine:
   Control settings:
      Displacement: 5 mm/min
      Maximum force: 500 N
      Cylindrical pressing rod
      Measurement of pressure (N/mm2) as a function of the deformation (%)
Calipers.

The protocol followed during the course of this testing is as follows:
Measurement of the thickness of the “test pieces” with the aid of the caliper:

| | Thickness (mm) | | | |
|---|---|---|---|---|
| | 1^{st} | 2^{nd} | 3^{rd} | |
| Identification | measurement | measurement | measurement | Average |
| A | 8.23 | 7.88 | 7.95 | 8.02 |
| B | 7.70 | 7.60 | 7.55 | 7.62 |
| C | 7.22 | 7.82 | 8.05 | 7.70 |
| D | 7.60 | 7.36 | 7.32 | 7.43 |

Measurement of the hardness of the “test pieces” with the aid of the JFC-TC3 machine.

The following results were obtained:
Measurement of thickness of the “test pieces”:

| | Thickness (mm) | | | |
|---|---|---|---|---|
| | 1^{st} | 2^{nd} | 3^{rd} | |
| Identification | measurement | measurement | measurement | Average |
| A | 8.23 | 7.88 | 7.95 | 8.02 |
| B | 7.70 | 7.60 | 7.55 | 7.62 |
| C | 7.22 | 7.82 | 8.05 | 7.70 |
| D | 7.60 | 7.36 | 7.32 | 7.43 |

Measurement of hardness of the “test pieces”:

| | Stress (MPa) | | | | Deformation (mm) | | | |
|---|---|---|---|---|---|---|---|---|
| | 1^{st} | 2^{nd} | 3^{rd} | | 1^{st} | 2^{nd} | 3^{rd} | |
| Identification | measurem't | measurem't | measurem't | Avg. | measurem't | measurem't | measurem't | Avg. |
| A | 488.2 | 489.0 | 488.1 | 488.4 | 0.45 | 0.42 | 0.48 | 0.45 |
| B | 489.1 | 489.1 | 488.8 | 489.0 | 0.53 | 0.54 | 0.44 | 0.5 |
| C | 489.8 | 488.7 | 487.7 | 488.7 | 0.45 | 0.54 | 0.53 | 0.5 |
| D | 488.3 | 488.9 | 488.3 | 488.5 | 0.48 | 0.39 | 0.72 | 0.53 |

| | | | |
|---|---|---|---|
| General average | 488.7 | General average | 0.5 |

The product, once hardened, resists pressure on the order of 480 Mp.

### EXAMPLE 4

### Osteogenicity And Biocompatibility

a) Implants in Rabbits.

The objectives of these implants are to verify that the compound according to the invention does not induce an inflammatory reaction inappropriate for bone reconstruction, to with, no fibrosis installed and start² of bone reconstruction. ² Translator's Note: The word “déut” is misspelled in the original. Changed in the translation.

6 implants of the compound according to Example 1 were made in adult rabbits, for a 4-week period, with allogenic bone.

The conclusions of the test were that the tolerance is compatible.

b) Implants in Sheep

24 implants of the compound according to the invention were made in sheep, for a 12-week period, with xenogenic bone (diameter 4.5 mm).

A histological study of bone density and cell infiltration was then carried out. Density and homogeneity are highly satisfactory.

c) Biocompatibility

The sensitization tests on guinea pigs (evaluation, across 38 guinea pigs, of cytotoxicity upon extraction (evaluated on cell series of mice), of acute systematic toxicity (evaluated on 10 mice in IV, 10 in IP) and the AMES test (evaluation of mutagenic power across five strains of salmonella) all gave results conforming to standard 10993.

1. Bone filling compound characterized by the fact that it includes allogenic bone powder, calcium sulfate, a binder chosen from starch or a starch derivative, and a mixing solution.

2. Compound according to claim 1, characterized by the fact that the starch derivative is chosen from among hydroxyethyl starch, carboxymethyl starch, starch glycolic acid, glycerol starch and pre-gelatinized starch.

3. Compound according to claim 1, characterized by the fact that the starch derivative is hydroxyethyl starch.

4. Compound according to any one of the foregoing claims characterized by the fact that the calcium sulfate is hemi-hydrated calcium sulfate, the defined crystalline form of which may be alpha, beta or both.

5. Compound according to any one of the foregoing claims characterized by the fact that the allogenic bone powder is chosen from among bone powders, whether human or not, whether de-mineralized or not, which have undergone a viroinactivation treatment, with a particle size between 200 and 1600 μm.

6. Compound according to any one of the foregoing claims characterized by the fact that the mixing solution is a physiologically compatible aqueous solution chosen from among an isotonic solution, physiological saline solution or a physiological liquid like serum or blood.

7. Compound according to any one of the foregoing claims characterized by the fact that the allogenic bone powder will be incorporated in proportions between 0.1 and 80% v/v, the calcium sulfate in proportions from 50 to 300% m/v, the hydroxyethyl starch from 0.1 to 20% v/v and the mixing solution from 0.3 to 80% v/v.

8. Use, for the filling of bone defects, of a bone filling compound, characterized by the fact that it includes allogenic bone powder, calcium sulfate, a binder chosen from starch or a starch derivative, and a mixing solution.

9. Kit made up of a container including a compound containing allogenic bone powder, calcium sulfate, a binder chosen from starch or a starch derivative, and a container that holds the mixing solution.

10. Kit according to the foregoing claim characterized by the fact that it also includes molds for osteotomy wedges.
